(19) United States
(12) Patent Application Publication
(10) Pub. No.: US 2021/0121432 A1
(43) Pub. Date: Apr. 29, 2021

(54) Bambuterol for the Treatment of Alzheimer's Disease

(71) Applicant: UNIVERSITE DE CAEN NORMANDIE, Caen (FR)

(72) Inventors: Christophe ROCHAIS, Caen (FR); Patrick DALLEMAGNE, Seulline (FR); Anne-Claire GROO, Eterville (FR); Aurélie MALZERT-FRÉON, Amayé sur Seulles (FR)

(73) Assignee: Université de Caen Normandie, Caen (FR)

(21) Appl. No.: 17/045,402

(22) PCT Filed: Apr. 16, 2019
(86) PCT No.: PCT/IB2019/000523

§ 371 Date: Oct. 5, 2020

Related U.S. Application Data
(60) Provisional application No. 62/658,631, filed on Apr. 17, 2018.

(51) Int. Cl. A61K 31/27; A61P 25/28

(52) U.S. Cl. CPC A61K 31/27; A61P 25/28

(57) ABSTRACT

Bambuterol for the Treatment of Alzheimer's Disease Bambuterol or the pharmaceutically acceptable salt thereof is used in an effective amount to treat Alzheimer's disease. The compound is administered intranasal, or through oral or intravenous routes and under an appropriate formulation for getting around the blood brain barrier, wherein it may do one more of preventing, slowing the progression of, or reversing Alzheimer's disease.

### FIELD OF THE INVENTION

The present invention relates to the field of the treatment of Alzheimer's disease.

### BACKGROUND OF THE INVENTION

Tens of millions of people, most of whom are over the age of 65, are afflicted with Alzheimer's disease, which is a progressive and debilitating neurological disorder. The disease appears in both humans and other mammals, and subjects who are afflicted with it may present with one or more of dementia, loss of memory, decline in thinking, decline in language, decline in learning capacity, and disorientation. Additionally, they may have challenges and/or deficits with respect to visual spatial skills.

Researchers have been aware for some time that the β-amyloid (“Aβ”) peptide has a toxic effect on persons, particularly when in the form of small, soluble oligomeric Aβ aggregates and that it is prevalent in subjects who are afflicted with Alzheimer's disease. Aβ peptides are known to be produced as soluble monomers that undergo oligomerization and amyloid fibril formation via a nucleation-dependent process. When Aβ fibril formation occurs in vitro, various nonfibrillar intermediately aggregates are generated. These aggregates are collectively referred to as soluble oligomers and protofibrils, and they have been shown to precede the appearance of fibrils. Therefore, these protofibrils are an attractive target for treating, preventing, and managing Alzheimer's disease.

Currently, there is a lack of satisfactory means to address the toxicity of the aforementioned protofibrils and similarly a lack of satisfactory treatment options for patients who have, or are at risk for having, Alzheimer's disease. Thus, there is a need for compositions and methods to do one or more of prevent, manage, and treat Alzheimer's disease. The present invention is directed to this need.

### BRIEF DESCRIPTION OF THE FIGURES

FIGS. 1A to 1H are representations of photographs of cells that have been stained.

FIG. 2A is a bar graph that shows the effect of bambuterol on the survival of MAP-2 neurons. FIG. 2B is a bar graph that shows the effect of bambuterol on a total neurite network. FIG. 2C is a bar graph that shows the effect of bambuterol on neurite length normalized buy the number of neurons. FIG. 2D is a bar graph that shows the effect of bambuterol on the phosphorylation of Tau (AT100) in MAP-2 neurons in a primary culture of hippocampal neurons. Donepezil was used as a positive control. Results are expressed as a percentage of control as mean±SEM (n=4-6/group).

FIG. 3 is a bar graph that shows the effect of bambuterol on the number of synapses in a primary culture of hippocampal neurons. Donepezil served as a positive control. Results are expressed as a percentage of control as mean±SEM (n=4-6/group).

### SUMMARY OF THE INVENTION

The present invention provides compositions and methods for the prevention, management, and treatment of Alzheimer's disease. Through the use of these compositions and methods, one may do one or more of lessen the severity of, or reverse the conditions of or prevent the occurrence of Alzheimer's disease.

According to a first embodiment, the present invention provides a method of preventing, managing, or treating Alzheimer's disease comprising administering an intranasal formulation comprising a therapeutically effective amount of bambuterol or a pharmaceutically acceptable salt thereof. In some embodiments, the active ingredient (bambuterol or its pharmaceutically acceptable salt) is contained in liposomes or mixed with phospholipids or micelles. For example, the bambuterol or its pharmaceutically acceptable salt may be encapsulated in liposomes to form an encapsulated therapeutic delivery composition. This encapsulated therapeutic delivery composition may, for example, be integrated in a gel such as a thermoresponsive gel. In other embodiments, the bambuterol or its pharmaceutically acceptable salt is not encapsulated and is free within a formulation such as a liquid, gel, or other composition.

When administering bambuterol through an intranasal formulation, one may include one or more membrane disruptive fatty acids such as dideconoylphosphatidylcholine and lysophosphatidylcholine, and one or more membrane disruptive surfactants such as sodium lauryl sulphate, saponin, and poloxyethylene-9-lauryl ether. Further in some embodiments, one may additionally or alternatively include bile salts such as sodium deoxycholate, sodium glycocholate or sodium taurodihydrofusidate. Similarly, in some embodiments, one may additionally or alternatively include enzyme inhibitors, e.g., one or both of bestatin and amastatia. Still further, when the formulation is a gel, optionally one may include a bioadhesive material such as one or more of carbopol, starch microspheres, or chitosan.

According to a second embodiment, the present invention provides a pharmaceutical composition comprising a liposome, wherein the liposome encapsulates bambuterol or a pharmaceutically acceptable salt thereof. Preferably, a sufficient amount of the bambuterol or a pharmaceutically acceptable salt thereof is present in a plurality of liposomes in a formulation that is capable of causing or contributing to the desired therapeutic effect, i.e., prevention, management, or treatment of Alzheimer's disease.

According to a third embodiment, the present invention provides bambuterol or a pharmaceutically acceptable salt thereof for use in the treatment of Alzheimer's disease.

According to a fourth embodiment, the present invention provides a method of preventing, managing, or treating Alzheimer's disease comprising: administering an oral formulation comprising a therapeutically effective amount of bambuterol or a pharmaceutically acceptable salt thereof.

According to a fifth embodiment, the present invention provides a method of preventing, managing, or treating Alzheimer's disease comprising: administering intravenously a therapeutically effective amount of bambuterol or a pharmaceutically acceptable salt thereof.

Through the various embodiments of the present invention, one may effectively and efficiently do one or more of treating, managing, or preventing Alzheimer's disease. Further, a number of the various embodiments of the present invention allow for efficient delivery of bambuterol or its pharmaceutically acceptable salt through, for example, the nasal passageway for delivery across the blood-brain barrier and to the central nervous system (CNS).

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided for the convenience of the reader and do not limit the scope of any of the embodiments disclosed herein.

### Definitions

Unless otherwise stated or implicit from context the following terms and phrases have the meanings provided below.

The articles “a” and “an” refer to one or more than one of the noun that follows.

The terms “administer” and “administration” refer to the act of physically delivering a substance as it exists outside of the body into a subject. Unless otherwise specified, administration includes all forms known in the art for delivering therapeutic agents, including but not limited to oral, topical, mucosal, intradermal, intravenous, intramuscular, and intranasal delivery as well as other methods of physical delivery described herein or known in the art.

The term “bambuterol” refers to a molecule that has the formula (C₁₈H₂₉N₃O₅) and the structure of:

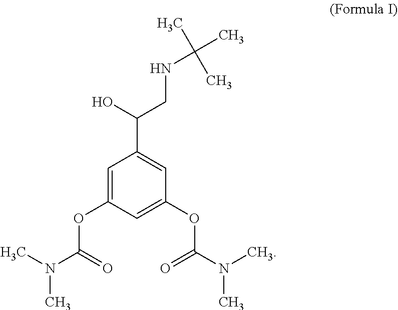 [SMILES: CN(C)C(=O)Oc1cc(OC(=O)N(C)C)cc(C(O)CNC(C)(C)C)c1]

Bambuterol may exist as represented in Formula I or as a salt such as a pharmaceutically acceptable salt. Examples of salts of bambuterol include but are not limited to the salts that it forms with any one or more of the following acids: hydrochloride, hydrobromide, sulphate, hydrogen sulphate, dihydrogen phosphate, methanesulphonate, methyl sulphate, acetate, oxalate, maleate, fumarate, succinate, 2-naphthalene-sulphonate, glyconate, gluconate, citrate, tartaric, lactic, pyruvic isethionate, benzenesulphonate, or para-toluenesulphonate. Additionally, bambuterol or its salts may be present as a pure R or S enantiomer, for example 100% pure or 90-99.99% pure or 95-99% pure, or as a racemic mixture.

The term “co-administer” means that a composition described herein is administered at the same time, prior to, or after the administration of one or more additional therapeutic compositions, including but not limited to an analgesic (e.g., acetaminophen) so as to allow the plurality of compounds to provide a benefit due to their introduction at the same time or in the sequence as introduced. Further, the terms “co-administration,” “in combination with,” and grammatical equivalents thereof are used interchangeably herein. Co-administration includes administering two active agents simultaneously, approximately simultaneously (e.g., within about 1, 5, 10, 15, 20, or 30 minutes of each other), or sequentially in any order. Thus, co-administration can include administering one active agent (e.g., a compound described herein) within 0.5, 1, 2, 4, 6, 8, 10, 12, 16, 20, or 24 hours of a second active agent. Co-administration can also be accomplished by co-formulation, e.g., preparing a single dosage form that includes both active agents. As persons of ordinarily skill in the art will recognize, even in a co-formulation, the active agents can initially be formulated separately. In such instances, the active agents are admixed and included together in the final form of the dosage unit. Alternatively, co-administration as described herein can include administering two separate unit dosage forms of at least two separate active agents. The term co-administration also encompasses administration of two or more agents to a subject so that both agents and/or their metabolites are present in the subject at the same time.

The term “continuous” means that a therapeutic compound, such as bambuterol or a pharmaceutically acceptable salt thereof, is administered at least daily for an uninterrupted period.

The term “daily” means that a therapeutic compound, such as bambuterol or a pharmaceutically acceptable salt thereof, is administered once or more than once each day for a period of time.

The term “cycling” means that a therapeutic compound, such as bambuterol or a pharmaceutically acceptable salt thereof is administered daily or continuously but with a rest period.

The phrases “effective amount” and “therapeutically effective amount” refer to an amount sufficient to prevent, treat, and/or manage the symptoms, neurological damage and/or underlying causes of a disease or disorder.

The term “encapsulated” means contained within or surrounded by such that an encapsulating material forms a barrier between a first substance and another substance or the environment.

The term “formulation” means a composition that comprises a therapeutically effective amount of an active ingredient. A formulation may also be referred to herein as a pharmaceutical composition, and may be a liquid, a solid, or a gel or any combination thereof. Further, it may, for example, be a mixture, a suspension, or solution, and it may include one or more additional ingredients that are or are not active.

The term “intranasal” refers to a route of delivery through the nasal cavity and/or through the nasal sinuses. Intranasal administration may, for example, be by spray, drops, powder, gel, film, inhalant, or other means. An “intranasal formulation” is a composition that is designed and that has the necessary components to allow for administration through the nasal cavity and/or through the nasal sinuses.

The term “intravenous” refers to the introduction of a composition into the vein of an organism such as a human through, for example, a needle and syringe or an infusion.

The terms “manage,” “managing,” and “management” refer to slowing the progression, spread, or worsening of a disease or disorder, or of one or more symptoms thereof. In certain cases, the beneficial effects that a subject derives from an agent that manages a disorder do not result in a cure of the disease or disorder.

The term “oral” refers to a route of administration wherein the dosage form is taken through the mouth. Thus, oral administration may be part of enteral administration, which also includes buccal (dissolved inside the cheek), sublabial (dissolved under the lip) and sublingual administration (dissolved under the tongue). Enteral medications (and thus medications that may be administered orally) come in various forms, including but not limited to: tablets to swallow, chew or dissolve in water or under the tongue; capsules and chewable capsules (with a coating that dissolves in the stomach or bowel to release the medication there); time-release or sustained-release tablets and capsules (which release the medication gradually); powders or granules; teas; drops; and liquid medications and syrups.

The terms “preventing” and “prevention” refer to the administration of a compound provided herein, with or without another additional active compound, prior to the onset of symptoms, particularly to patients at risk of developing Alzheimer's disease. For example, patients with familial history of the disease are candidates for preventive regimens. In addition, patients who have a history of recurring symptoms are also potential candidates for the prevention.

The terms “subject” and “patient” refer to an animal, including, but not limited to, a mammal, including but not limited to a primate (e.g., human), cow, sheep, goat, horse, dog, cat, rabbit, rat, or mouse. In various embodiments, subjects herein can be characterized by the disease being treated (e.g., “Alzheimer's disease”).

The phrase “therapeutic delivery composition” refers to a complex or other association of an active ingredient and one or more other moieties or components in order to facilitate delivery to a subject or to a specific tissue of a subject. By way of non-limiting examples, therapeutic delivery compositions can comprise active ingredients, e.g., bambuterol or a pharmaceutically acceptable salt thereof when it is associated with or encapsulated by a liposome.

The phrase “therapeutically effective amount” refers to the amount of a compound or substance that, when administered, is sufficient to prevent development of, or to alleviate to some extent, one or more of the symptoms of or to reverse the progression of or damage done by a disease or disorder, e.g., Alzheimer's disease. The phrase also refers to the amount of a compound that is sufficient to elicit the biological or medical response of a biological molecule (e.g., a protein, enzyme, RNA, or DNA), cell, tissue, system, animal, or human that is being sought by a researcher, veterinarian, medical doctor, or clinician. Furthermore, a therapeutically effective amount of a compound means an amount of a therapeutic agent, alone or in combination with other therapies that provides a therapeutic benefit in the treatment, prevention, or management of a disease, e.g., Alzheimer's disease.

The terms “treat,” “treating,” and “treatment” refer to alleviating or abrogating a disease or disorder, e.g., Alzheimer's disease, or one or more of the symptoms associated with the disease, or alleviating or eradicating the cause or causes of the disease or disorder itself.

Bambuterol

Bambuterol is a known molecule, and according to the present invention, one may use bambuterol or a pharmaceutically acceptable salt thereof to treat, manage, or prevent Alzheimer's disease. In some embodiments, one may use a formulation of which bambuterol or a pharmaceutically acceptable salt thereof is the sole active ingredient or in which bambuterol or a pharmaceutically acceptable salt thereof is co-administered in the same composition with another active ingredient. In other embodiments, the formulation comprising bambuterol or a pharmaceutically acceptable salt thereof may be co-administered with other active ingredients in different formulations and these other active ingredients may be separately administered at the same time or sequentially.

In some embodiments, the use of bambuterol has beneficial effects on neuronal survival and/or neurite networks such as within the hippocampus. In some of these embodiments, the bambuterol is able to cross the blood-brain barrier and is able to reduce or to nullify toxicity of one or more Aβ peptides, nonfibrillar intermediary aggregates of Aβ peptides, and Aβ fibrills. Additionally, bambuterol may be used to reduce the hyperphosphorylation of Tau.

In some embodiments, the bambuterol or a pharmaceutically acceptable salt thereof is part of an intranasal formulation. The intranasal formulation may, for example, be in the form of a spray, an ointment, drops, or a gel that is capable of being sprayed or that integrates the bambuterol or a pharmaceutically acceptable salt thereof, which may or may not be encapsulated in a liposome. Optionally, the formulation may contain one or more of water, organic solvents, excipients, stabilizers, preservatives, enzyme, inhibitors, and diluents.

Therapeutic Delivery Compositions

In some embodiments, the bambuterol or a pharmaceutically acceptable salt is in a form that it may be delivered to a subject such as a subject in need thereof. This form may, for example, be within a gel or liquid or other carrier but not encapsulated by or conjugated to another substance, and thus “free.” When free, the bambuterol or a pharmaceutically acceptable salt may be located, e.g., contained or housed or mixed within a gel or liquid and thus free to interact with other components of or contained in the liquid or gel. In these embodiments, although not encapsulated, the bambuterol or its pharmaceutically acceptable salt is still part of a therapeutic delivery composition

Alternatively, the bambuterol or a pharmaceutically acceptable salt may be encapsulated, e.g., in liposomes or mixed with phospholipids or micelles. For example, the bambuterol or its pharmaceutically acceptable salt may be encapsulated in liposomes to form an encapsulated therapeutic delivery composition. Through the use of liposomes, a therapeutically effective amount of bambuterol and/or its pharmaceutically acceptable salt may effectively and efficiently be delivered intranasally.

Liposomes are typically spherical vesicles that are formed by a membrane bilayer and may, by way of example, be composed of phospholipids. Liposomes have an aqueous core that enables them to encapsulate hydrophilic drugs. Some liposomes are thermosensitve and of particular use in some embodiments of the present invention. Examples of these liposomes include but are not limited to: 1,2-dipalmitoyl-sn-glycero-3-phosphocholine (DPPC) and 1,2-distearoyl-sn-glycero-3-phosphocholine (DSPC) 3:1 (mol/mol); DPPC/HSPC/Chol/DPPE-PEG 50:25:15:3 (mol/mol); DPPC/lyso-PC/DSPE-PEG2000 90:10:4 (mol/mol); DPPC/DSPC/DPPG2 50:20:30 (mol/mol); DPPC/DSPC/DSPE-PEG2000 80:15:5 (mol/mol); DPPC/Brij78 96:4 (mol/mol); and DPPC/DSPE-PEG2000/Chol/ELP 55:2:15:0.4125 (mol/mol). In some embodiments, the liposomes comprise, consist essentially of, or consist of soybean phosphatidylcholine and cholesterol in molar proportions (mol/mol) of 10:1 to 1:1, for example 10:1; 9:11; 8:1; 7:1; 6:1; 5:1; 4:1; 3:1; 2:1; or 1:1 or any proportion in between.

Within individual liposomes may be exclusively bambuterol or its pharmaceutically acceptable salt or that active ingredient with additional ingredients. Additionally, in some embodiments, within a formulation there may be a uniformity of phospholipid types and cores, a uniformity of phospholipid types but different cores, a plurality of different phospholipid types with a uniformity of cores, or a plurality of different phospholipid types and a plurality of different cores (with either each unique core ingredient associated with a different phospholipid type or a plurality of different phospholipid types associated with the same core and/or a plurality of different cores associated with the same phospholipid types).

Gels

In some embodiments, a therapeutic delivery composition is integrated in a gel. Thus, by way of a non-limiting example, an encapsulated therapeutic delivery composition (e.g., a liposome comprising, consisting essentially of, or consisting of phospholipids and bambuterol or its salt) may be integrated in a gel such as a thermoresponsive gel. In some embodiments, the formulation is liposomal such that the bambuterol and/or its pharmaceutically acceptable salt is contained within a liposomal structure and integrated into a thermoresponsive gel. These gels may, for example, have one or more physical forms including but not limited to slabs, films, in situ hydrogels, nano-gels, micro-particles and nanoparticles. To be integrated in a gel means being mixed or distributed regularly or irregularly in the gel. When integrated, the therapeutic delivery composition will retain association with the gel until physical dislodgement by, for example, movement or dissolution of the gel, which may, for example, be due to diffusion or a change in temperature, or a combination thereof.

In certain embodiments, the thermoresponsive hydrogel has a lower critical solution temperature (LCST) below body temperature. This type of thermoresponsive hydrogel remains fluid below physiological temperature (e.g., 37° C. for humans) or at or below room temperature (e.g., 25° C.), solidifies (into a hydrogel) at physiological temperature, and is biocompatible. For example, the thermoresponsive hydrogel may be a clear liquid at a temperature below 34° C., and reversibly solidify into a gelled composition at a temperature above 34° C. Generally, the LCST-based phase transition occurs upon warming in situ as a result of entropically-driven dehydration of polymer components, which leads to collapse of the polymer. Various naturally derived and synthetic polymers exhibiting this behavior may be utilized.

Natural polymers that one may use in some embodiments include elastin-like peptide and polysaccharide derivatives, while notable synthetic polymers include those based on poly(n-isopropyl acrylamide) (PNIPAAm), poly(N,N-dimethylacrylamide-co-N-phenylacrylamide), poly(glycidyl methacrylate-co-N-isopropylacrylamide), poly(ethylene oxide)-b-poly(propylene oxide)-b-poly(ethylene oxide), poly(ethylene glycol)-polyester copolymer, poly(ethylene glycol)-poly(serinol hexamethylene urethane), and amphiphilic block copolymers.

In certain embodiments, the amphiphilic block copolymer comprises, consists essentially of, or consists of a hydrophilic component selected from polyethylene oxide (PEO), polyvinyl alcohol (PVA), polyglycolic acid (PGA), poly (N-isopropylacrylamide), poly(acrylic acid) (PAA), poly vinyl pyrrolidone (PVP) or mixtures thereof, and a hydrophobic component selected from polypropylene oxide (PPO), poly(lactic acid) (PLA), poly(lactic acid co glycolic acid) (PLGA), poly (β-benzoyl L-aspartate) (PBLA), poly(γ-benzyl-L-glutamate) (PBLG), poly(aspartic acid), poly(L-lysine), poly(spermine), and poly(caprolactone), and mixtures thereof.

In certain embodiments, the hydrogel is non-biodegradable (e.g., PNIPAAm). In other embodiments, the hydrogel is biodegradable.

By way of example, NIPAAm-based polymers can be made by conjugating PNIPAAm with natural biodegradable segments such as MMP-susceptible peptide, gelatin, collagen, hyaluronic acid, and dextran. Copolymers formed from NIPAAm and monomers with degradable side chains comprise another category of NIPAAm-based bioabsorbable, thermoresponsive hydrogels that may be used in connection with various embodiments of the present invention. As persons of ordinary skill in the art will recognize, hydrolytic removal of hydrophobic side chains increases the hydrophilicity of the copolymer, raising the LCST above body temperature and making the polymer backbone soluble.

In a further embodiment, the thermoresponsive hydrogel degrades and dissolves under physiological conditions in a time-dependent manner. For example, the gel may comprise, consist essentially of or consist of a copolymer of one or more of N-isopropylacrylamide (NIPAAm) residues, hydroxyethyl methacrylate (HEMA) residues and methacrylate-polylactide (MAPLA) macromer residues. Alternately, the copolymer comprises, consists essentially of, or consists of N-isopropylacrylamide residues, acrylic acid (AAc) residues, and hydroxyethyl methacrylate-poly(trimethylene carbonate) (HEMAPTMC) macromer residues.

Additional biodegradable hydrogels that may be of use in various embodiments of the present invention include, but are not limited to, albumin, heparin, poly(hydroxyethylmethacrylate), fibrin, carboxymethyl cellulose, hydroxypropylmethyl cellulose, lectin, polypeptides, agarose, amylopectin, carrageenan, chitin, chondroitin, lignin, hylan, α-methyl galactoside, pectin, starch, and sucrose. Additionally, the hydrogel may be made from a combination or mixture of any of the hydrogels disclosed herein.

In some embodiments, regardless of whether liposomes or gels are used, the formulation may comprise water and one or more pharmaceutically acceptable carriers or excipients. Preferably, these additional components are non-toxic solid, semisolid, or liquid fillers, diluents, encapsulating material or formulation auxiliaries.

Water as well as other solvents, solubilizing agents and emulsifiers suitable for use in place of or in addition to it, include but are not limited to, saturated aliphatic mono- and polyvalent alcohols that contain 2-6 carbon atoms (including, but not limited to, ethanol, 1,2-propylene glycol, sorbitol, and glycerine), polyglycols such as polyethylene glycols, and surfactants/emulsifiers like the fatty acid esters of sorbitan, and mixtures thereof. Oils, in particular, cottonseed, peanut, or corn oils, may also be added to the compositions. The combination of the additional solvents in an aqueous solution should preferably not exceed about 15% (w/v) of the total composition.

Certain liquid compositions of the present invention may further comprise one or more preservatives and/or one or more stabilizers. Preservatives that are suitable for use in various embodiments of the present invention include, but are not limited to, edetic acid and its alkali salts such as disodium EDTA (also referred to as “disodium edetate” or “the disodium salt of edetic acid”) and calcium EDTA (also referred to as “calcium edetate”), benzyl alcohol, methylparaben, propylparaben, butylparaben, chlorobutanol, phenylethyl alcohol, benzalkonium chloride, thimerosal, propylene glycol, sorbic acid, and benzoic acid derivatives. The preservatives may be used at a concentration of from about 0.001% to about 0.5% (w/v) in the final composition. Certain compositions of the present invention may further comprise one or more solubility-enhancing agents. Solubility-enhancing agents that are suitable for use in the compositions of the present invention include, but are not limited to, polyvinylpyrrolidone (preferably grades 25, 30, 60, or 90), poloxamer, polysorbate 80, sorbitan monooleate 80, and polyethylene glycols (molecular weights of 200 to 600).

Certain compositions of the present invention may further comprise one or more agents that are used to render the composition isotonic, particularly in those compositions in which water is used as a solvent. Such agents are particularly useful in compositions formulated for nasal or ocular applications, because they adjust the osmotic pressure of the formulations to the same osmotic pressure as nasal or ocular secretions. Agents that are suitable for such a use in the compositions of the present invention include, but are not limited to, sodium chloride, sorbitol, propylene glycol, dextrose, sucrose, and glycerine, and other isotonicity agents that are known in the art.

In some embodiments, the gel is mucoadhesive. As persons of ordinary skill in the art are aware, mucoadhesion refers to the adhesion between two materials, at least one of which is a mucosal surface. Examples of mucoadhesive substances include but are not limited to, gels that comprise lectins, invasins, and fimbrial proteins.

In some embodiments, it is desirable that the formulations of the present invention have a pH of about 4.5 to about 7.4, and preferably have a pH of about 5.5 to 7.1. Accordingly, in some embodiments, the formulations of the present invention may further comprise one or more buffering agents or combinations thereof that are used to adjust the compositions into and/or maintain the compositions within the desired pH range. Adjustment of pH or buffering agents that are suitable for use in the compositions of the present invention include, but are not limited to, citric acid, sodium citrate, sodium phosphate (dibasic, heptahydrate form), and boric acid or equivalent conventional buffers, or combinations thereof.

Liquid Sprays and Drops

As noted above, within the scope of the present invention is also the administration of bambuterol or its pharmaceutically acceptable salt without the use of gels. For example, one may administer bambuterol or its pharmaceutically acceptable salt as a liquid in the form of a spray or drops. In some embodiments, the amount of bambuterol or a pharmaceutically acceptable salt thereof in the formulation is a therapeutically effective amount and may be measured by weight per volume (w/v). In some embodiments, the bambuterol or a pharmaceutically acceptable salt thereof is present in an amount about 0.01% to about 1.0% (w/v) or about 0.5% to about 0.5% (w/v), or about 0.1% to about 0.5% (w/v) or about 0.1% to about 0.4% (w/v).

When the formulation is a liquid spray or a drop, any one or more of the additional ingredients referenced in this disclosure as being useful with or without gels may be used. Additionally and particularly when using sprays, it may be advantageous to include one or more propellants. When propellants are used, suitable ones include, but are not limited to, hydrocarbons particularly 1,1,1,2-tetrafluoroethane (HFA134a) and 1,1,1,2,3,3,3-heptafluoropropane (HFA227) or mixtures of them.

Delivery and Dosing

The pharmaceutical compositions of the present invention may be delivered to a subject in need thereof for the purpose of one or more of preventing, treating, or managing Alzheimer's disease. In some embodiments, the desired effect is accomplished by administering bambuterol or a pharmaceutically acceptable salt thereof to a subject by dispensing drops, spraying a formulation, or depositing a gel. The subject may have Alzheimer's disease, regardless of whether having been diagnosed as having it or may be or believe himself or herself to be at risk for having Alzheimer's disease.

When the formulation is a liquid that is in the form of drops or a spray, delivery may be through known techniques for dispensing those respective forms of liquids, and these liquids may be dispensed at regular intervals, for example, once per day, twice per day or three times per day, or at irregular intervals.

Each of the drops formulations and the sprays formulations may be housed within an intranasal delivery system. By way of a non-limiting example, the intranasal delivery system may comprise a bottle and a pump, e.g., a multi-dose pump.

When using a spray, one may, for example, administer the formulation through an intranasal delivery system that comprises a bottle that may be squeezed in order to dispense product. In some embodiments, systems for delivering nasal sprays comprise a chamber, a piston, and an actuator. Additionally, when a spray is used, in some embodiments, the intranasal delivery system delivers a volume of the composition of about 0.05 ml to about 1.0 ml per spray or about 0.1 ml to about 0.5 ml per spray. Further, by way of non-limiting examples, each dose may consist of one spray, one spray per nostril, or 2 to 4 sprays per nostril. When drops are used, in some embodiments, one to six drops are administered in each nostril during each administration period.

When a gel is used, the gel is inserted in the nasal cavity where, due at least in part to the viscosity of the gel and its components, the bambuterol (or its salt(s)), which may be contained in liposomes, are released at a desirable rate over a sufficiently long period that a subject tolerates the periodic insertion of new gels at regular or irregular intervals while also receives sufficient medication. In some embodiments, a new gel is inserted every 1 to 30 days or every 3 to 25 days or every 5 to 15 days, over a period of at least two months, at least six months, or at least one year. Within each gel, there may, for example, be 1 to 50 mg or 5 to 40 mg or 10 to 30 mg of the active ingredient.

Alternatively or additionally, the bambuterol (or its pharmaceutically acceptable salt) may be administered orally. Thus, the bambuterol (or its pharmaceutically acceptable salt) may, for example, be delivered in the form or capsule, tablet, caplet, gel, dissolvable strip, liquid drop, or other oral delivery form (e.g., powders, granules, teas, and syrups) that is now known or that comes to be known and that a person of ordinary skill in the art would recognize as useful in connection with the present invention. When administered through the oral cavity, the bambuterol may be absorbed there or it may enter the digestive tract and be absorbed into the blood stream from one or more points along the digestive tract.

Alternatively or additionally, the bambuterol may be administered intravenously. Thus, the bambuterol (or its pharmaceutically acceptable salt) may, for example, be delivered through an intravenous (“IV”) injection or through an intravenous infusion. In some embodiments, the bambuterol may be dissolved in saline prior to IV administration.

Alternatively or additionally, the bambuterol (or its pharmaceutically acceptable salt) may be in the form of a solution, e.g., an aqueous solution. To the solution, a person of ordinary skill in the art may add (by techniques now known or that come to be known to persons of ordinary skill in the art), one or more of excipients, surfactants, diluents, additional active ingredients or other substances that are now known or that come to be known for administering or preparing substances for delivery.

Regardless of the source of delivery, in some methods one delivers between 256 micrograms to 8 mg per kg per day or 512 micrograms to 4 mg per kg per day or 1 mg to 4 mg per kg per day. Persons of ordinary skill in the art can use the technologies now known or that come to be known to control time administration of the active ingredient at different frequencies in order to achieve these levels. Effective doses may also be extrapolated from dose-response curves derived from in vitro or animal model test systems. Such animal models and systems are well-known in the art.

As noted herein, various methods of the present invention deliver the therapeutic agent to the nasal cavity of a mammal. In some embodiments, it is preferred that the agent be delivered to the olfactory area in the upper one-third of the nasal cavity and, particularly, to the olfactory neuroepithelium in order to promote rapid and efficient delivery of the agent to the central nervous system. In some embodiments, delivery is along the olfactory neural pathway rather than the capillaries within the respiratory epithelium.

In some embodiments, the benefits of the present invention are realized when the concentration of bambuterol or the pharmaceutically acceptable salt thereof in the patient's brain is within the range of about 0.1 nM to about 50 M or about 1.0 nM to about 25 M or about 1 M to 10 M or about 1.0 nM to 1 M or about 1.0 nM to 50 nM.

Uses

Administration of the formulations may have one or more beneficial effects, including but not limited to inhibition of memory loss, inhibition of spatial memory loss, managing, i.e., slowing the progression of Alzheimer's disease, preventing Alzheimer's disease, treating Alzheimer's disease, or reducing the amount of Aβ or amyloid plaques or protein or oligomers comprising or derived from Aβ or amyloid proteins or plaques.

Additionally, various embodiments of the present invention are directed to the use of a bambuterol or a pharmaceutically acceptable salt thereof for the manufacture of a medicament for the treatment of Alzheimer's disease. These uses may be for treating, managing, or preventing Alzheimer's disease, and the medicament may be a formulation as described in any embodiments of the present invention and administered through a device described in connection with any of those embodiments.

Additional Active Ingredients

The bambuterol (or its salts) may be co-administered with other active ingredients. The co-administration may be within the same formulation, simultaneously within a different formulation via the same or different routes of administration, or sequentially within a different formulation via the same or different routes of administration.

Examples of additional active agents include, but are not limited to, additional antihistamines (including H1, H3 and H4 receptor antagonists), steroids (e.g., safe steroids), leukotriene antagonists, prostaglandin D2 receptor antagonists, decongestants, expectorants, anti-fungal agents, triamcinolone and triamcinolone derivatives, non-steroidal immunophilin-dependent immunosuppressants (NsIDIs), anti-inflammatory agents such as non-steroidal anti-inflammatory agents (NSAIDs), COX-2 inhibitors, anti-infective agents, mucolytic agents, anticholinergic agents, mast cell stabilizers, non-antibiotic anti-microbial agents, anti-viral agents, antiseptics, neurokinin antagonists, platelet activating factor (PAF), 5-lipoxygenase (5-LO) inhibitors, vitamins, and siRNA.

Examples of specific additional ingredients include acrivastine, azelastine, cyclizine, carebastine, cyproheptadine, carbinoxamine, doxylamine, dimethindene, ebastine, epinastine, efletirizine, ketotifen, levocabastine, mizolastine, mequitazine, mianserin, noberastine, meclizine, norastemizole, olopatadine, picumast, tripelenamine, temelastine, trimeprazine, triprolidine, bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine, pyrilamine, astemizole, terfenadine, loratadine, cetirizine, levocetirizine, fexofenadine, descarboethoxyloratadine, desloratadine, dimenhydrinate and hydroxyzine.

Examples of H3 receptor antagonists suitable for use in combination with bambuterol include, but are not limited to, thioperamide, impromidine, burimamide, clobenpropit, impentamine, mifetidine, clozapine, S-sopromidine, R-sopromidine, and ciproxifam.

Examples of leukotriene antagonists (e.g., leukotriene D4 antagonists) suitable for use in combination with bambuterol include, but are not limited to, albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast (e.g., montelukast sodium), moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, and zanamivir.

Examples of anti-fungal agents suitable for use in combination with bambuterol include, but are not limited to, amphotericin B, nystatin, fluconazole, ketoconazole, terbinafine, itraconazole, imidazole, triazole, ciclopirox, clotrimazole, and miconazole.

Examples of NSAIDs suitable for use in combination with bambuterol include, but are not limited to, ibuprofen, aceclofenac, diclofenac, naproxen, etodolac, flurbiprofen, fenoprofen, ketoprofen, suprofen, fenbufen, fluprofen, tolmetin sodium, oxaprozin, zomepirac, sulindac, indomethacin, piroxicam, mefenamic acid, nabumetone, meclofenamate sodium, diflunisal, flufenisal, piroxicam, ketorolac, sudoxicam, and isoxicam.

By “non-steroidal immunophilin-dependent immunosuppressant” or “NsIDI” is meant any non-steroidal agent that decreases proinflammatory cytokine production or secretion, binds an immunophilin, or causes down regulation of the proinflammatory reaction. NsIDIs suitable for inclusion in the present invention include, but are not limited to, calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

Examples of COX-2 inhibitors suitable for use in combination with bambuterol include, but are not limited to, rofecoxib, celecoxib, valdecoxib, lumiracoxib, meloxicam, and nimesulide.

Examples of steroids suitable for use in combination with bambuterol include but are not limited to, fluoromethalone, fluticasone, mometasone, triamcinolone, betamethasone, flunisolide, budesonide, beclomethasone, budesonide, rimexolone, beloxil, prednisone, loteprednol, dexamethasone and its analogues (e.g., dexamethasone beloxil).

Examples of anticholinergics suitable for use in combination with bambuterol include, but are not limited to, ipratropium, atropine, and scopolamine.

Examples of neurokinin antagonists suitable for use in combination with bambuterol include, but are not limited to, oximes, hydrazones, piperidines, piperazines, aryl alkyl amines, hydrazones, nitroalkanes, amides, isoxazolines, quinolines, isoquinolines, azanorbornanes, naphthyridines, and benzodiazepines.

Examples of 5-lipoxygenase (5-LO) inhibitors suitable for use in combination with bambuterol include, but are not limited to, zileuton, docebenone, piripost and tenidap.

The amounts of additional active agents (e.g., one or more steroid(s), leukotriene antagonist(s), antihistamine(s), decongestant(s), NSAIDs, etc.), can be present in any amount, for example about 0.01% to about 99% (e.g., about 0.01%, about 0.1%, about 1%, about 10%, about 20%, about 30%, about 40%, about 50%, about 60%, about 70%, about 80%, or about 90%).

Any of the features of the various embodiments described herein can be used in conjunction with features described in connection with any other embodiments disclosed unless otherwise specified. Thus, features described in connection with the various or specific embodiments are not to be construed as not suitable in connection with other embodiments disclosed herein unless such exclusivity is explicitly stated or implicit from context.

### EXAMPLES

In the examples below, the following experimental protocols were used.

Bambuterol, referred to as MR36621 in the experiments was tested on rat primary hippocampal neurons that were injured with Aβ. Donezpil was used as a reference test compound.

Primary Culture of Hippocampal Neurons

Rat hippocampal neurons were cultured as described by Callizot et al., Operational dissection of β-amyloid cytopathic effects of cultured neurons, J. Neurosci. Res. 2013 May; 91(5): 706-16. Pregnant female rats of 17 days gestation (Rats Wistar; Janvier Labs France) were euthanized using a deep anesthesia with a CO₂ chamber followed by a cervical dislocation. Fetuses were removed from the uterus and immediately placed in ice-cold L15 Leibovitz medium with a 2% penicillin (10,000 U/ml) and streptomycin (10 mg/ml) solution (PS) and 1% bovine serum albumin (BSA).

Hippocampal neurons were treated for 20 min at 37° C. with a trypsin-EDTA solution at a final concentration of 0.05% trypsin and 0.02% EDTA. The dissociation was stopped by adding Dulbecco's modified Eagle's medium (DMEM) with 4.5 g/liter of glucose, containing DNAse I grade II (final concentration 0.5 mg/ml) and 10% fetal calf serum (FCS). Cells were mechanically dissociated by three forced passages through the tip of a 10-ml pipette and then centrifuged at 515×g for 10 min at 4° C.

The supernatant was discarded, and the pellet was resuspended in a defined culture medium consisting of Neurobasal medium with a 2% solution of B27 supplement, 2 mmol/liter of L-glutamine, 2% of PS solution, and 10 ng/ml of brain-derived neurotrophic factor (BDNF). Viable cells were counted in a Neubauer cytometer, using the trypan blue exclusion test. Cells were seeded at a density of 20,000 per well in 96-well plates precoated with poly-L-lysine and cultured at 37° C. in an air (95%)-CO₂ (5%) incubator. The medium was changed every 2 days.

Test Compound and Human Aβ1-42 Exposure

The hippocampal neurons were exposed with A solutions after 17 days of culture. An Aβ1-42 preparation was done following the procedure described by Callizot et al., 2013. Aβ1-42 is a forty-two amino acid form of amyloid β in cerebrospinal fluid and is recognized by persons of ordinary skill in the art as a biomarker for Alzheimer's disease.

Aβ1-42 peptide was dissolved in the defined culture medium mentioned above, at an initial concentration of 40 μM. This solution was gently agitated for 3 days at 37° C. in the dark and immediately used after being properly diluted in culture medium to the concentrations used (20 μM (plate 13) or 2.5 μM (plate 14) corresponding to 2 μM or 0.25 μM of oligomers (AβO) respectively).

Bambuterol was placed in a culture medium and preincubated for 1 hour before Aβ application. An Aβ1-42 preparation was added to a final concentration of 20 or 2.5 μM (=2 μM or 0.25 μM of AβO, evaluated by automatic WB) diluted in control medium in presence of the compound.

Table I identifies two plates that exposed two concentrations of Aβ to different concentrations of bambuterol. The bambuterol was preincubated 1 hour before the application of Aβ.

TABLE I

| PLATE A (MAP-2/Tau) | PLATE B (PSD95/SYN) |
|---|---|
| Control (vehicle) | Control (vehicle) |
| +Aβ (20 μM 24 H)/vehicle | +Aβ (2.5 μM 24 H)/vehicle |
| +Aβ (20 μM 24 H)/MR36621 (1 nM) | +Aβ (2.5 μM 24 H)/MR36621 (1 nM) |
| +Aβ (20 μM 24 H)/MR36621 (5 nM) | +Aβ (2.5 μM 24 H)/MR36621 (5 nM) |
| +Aβ (20 μM 24 H)/MR36621 (10 nM) | +Aβ (2.5 μM 24 H)/MR36621 (10 nM) |
| +Aβ (20 μM 24 H)/MR36621 (50 nM) | +Aβ (2.5 μM 24 H)/MR36621 (50 nM) |
| +Aβ (20 μM 24 H)/MR36621 (100 nM) | +Aβ (2.5 μM 24 H)/MR36621 (100 nM) |
| +Aβ (20 μM 24 H)/MR36621 (500 nM) | +Aβ (2.5 μM 24 H)/MR36621 (500 nM) |
| +Aβ (20 μM 24 H)/MR36621 (1 μM) | +Aβ (2.5 μM 24 H)/MR36621 (1 μM) |
| +Aβ (20 μM 24 H)/Donepezil (1 μM) | +Aβ (2.5 μM 24 H)/Donepezil (1 μM) |

Plate A: Survival, Neurite Network and Phospho Tau Evaluation

Twenty-four hours after intoxication, the hippocampal neurons were fixed by a cold solution of ethanol (95%) and acetic acid (5%) for 5 min at −20° C. After permeabilization with 0.1% of saponin, cells were incubated for 2 hours with:
(a) chicken polyclonal antibody anti microtubule-associated-protein 2 (MAP-2), diluted at 1/1000 in PBS containing 1% fetal calf serum and 0.1% of saponin (this antibody allows the specific staining of neuronal cell bodies and neurites; and allow the study of neuronal cell death and neurite network); and
(b) mouse monoclonal antibody anti-phospho Tau (AT100) at dilution of 1/400 in PBS containing 1% fetal calf serum and 0.1% of saponin.

These antibodies were revealed with Alexa Fluor 488 goat anti mouse IgG and Alexa Fluor 568 goat anti-chicken IgG at the dilution 1/400 in PBS containing 1% FCS, 0.1% saponin, for 1 hour at room temperature.

For each condition, multiple pictures (representative of the whole well area) per well were taken using ImageXpress (Molecular Devices) at 20× magnification. All images were taken with the same acquisition parameters. Analyses were performed automatically by using Custom Module Editor® (Molecular Devices).

The following endpoints were assessed:
total number of neurons (neuron survival, number of MAP-2 positive neurons);
neurite network (in μm of MAP-2 positive neurons); and
area of hyperphosphorylated tau (AT100) in neurons (μm², Tau signal overlapping with MAP-2 positive neurons).

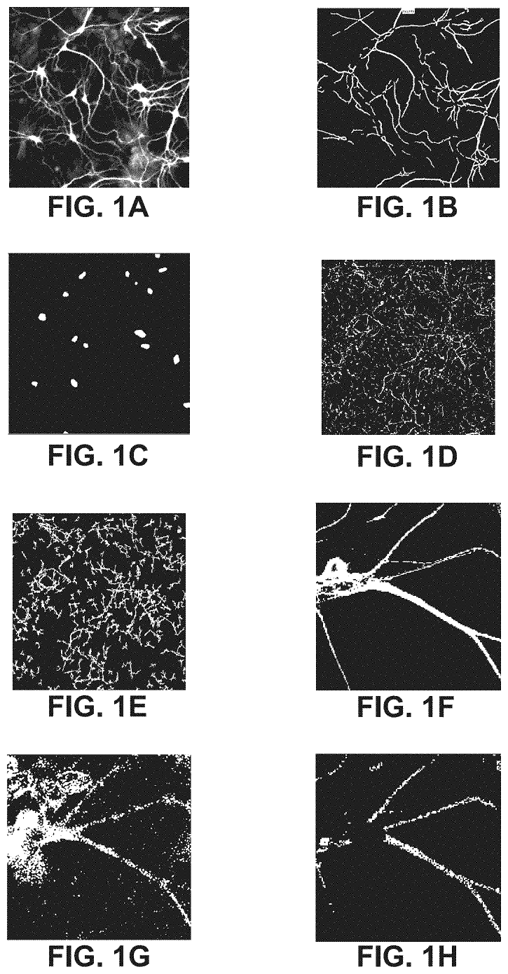

FIG. 1A is a representation of an original photograph with MAP-2 staining. FIG. 1B is a representation of a neurite network (MAP-2) segmentation. FIG. 1C is a representation of a number of MAP-2 positive cells segmentation.

FIG. 1D is a representation of an original photograph with AT100 staining. FIG. 1E is a representation of tau phosphor on Thr212 and Ser214 (AT100) segmentation.

Plate B: Synapses Evaluation

Twenty-four hours after intoxication, the hippocampal neurons were fixed by a cold solution of ethanol (95%) and acetic acid (5%) for 5 min at −20° C. After permeabilization with 0.1% of saponin, cells were incubated for 2 hours with:
(a) mouse monoclonal antibody anti post synaptic density 95 kDa (PSD95) at a dilution of 1/100 in PBS containing 1% fetal calf serum and 0.1% of saponin; and
(b) rabbit polyclonal antibody anti-synaptophysin (SYN) at the dilution of 1/100 in PBS containing 1% fetal calf serum and 0.1% of saponin.

These antibodies were revealed with Alexa Fluor 488 goat anti mouse IgG and Alexa Fluor 568 goat anti-rabbit IgG at the dilution 1/400 in PBS containing 1% FCS, 0.1% saponin, for 1 hour at room temperature.

For each condition, multiple pictures per well were taken using ImageXpress (Molecular Devices) with 40× magnification. All images were taken with the same acquisition parameters.

Synapses evaluation were performed automatically by using Custom module Editor® (Molecular Devices).

The following endpoints were assessed:
Total number of synapses (overlapping between PSD95/SYN).

FIG. 1F is a representation of another original photograph with MAP-2 staining. FIG. 1G is a representation of an original photograph of the images of FIG. 1F but with PSD95 staining. FIG. 1H is a representation of a segmentation of synapsis on neurites (PSD95/MAP-2).

Effects of Bambuterol on the Survival and Neurite Network of MAP-2 Hippocampal Neurons and on the Hyperphosphorylation of Tau.

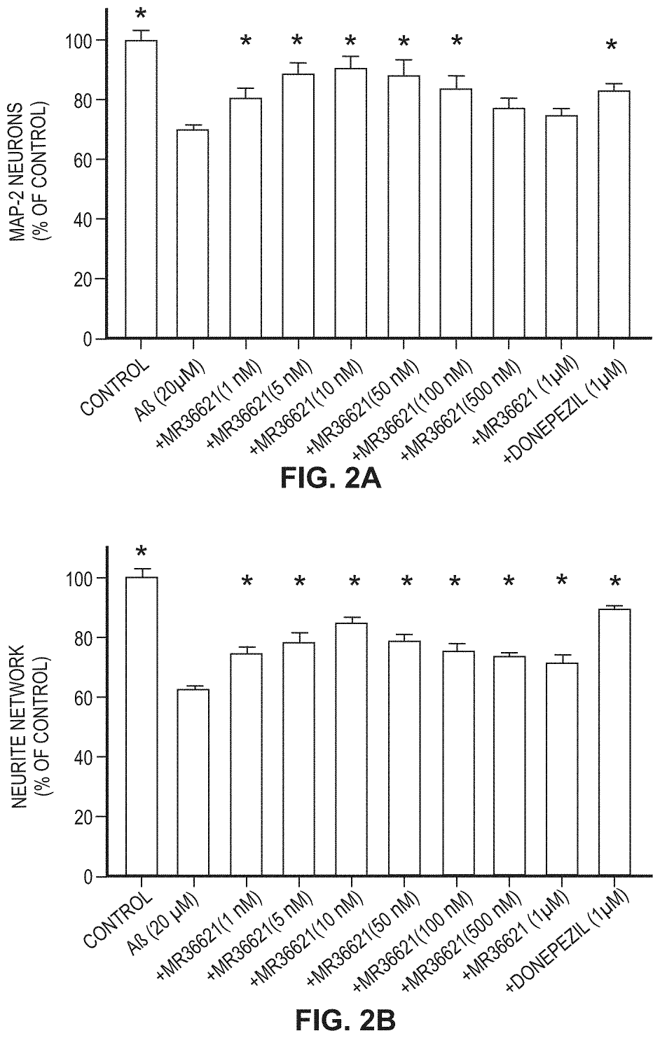

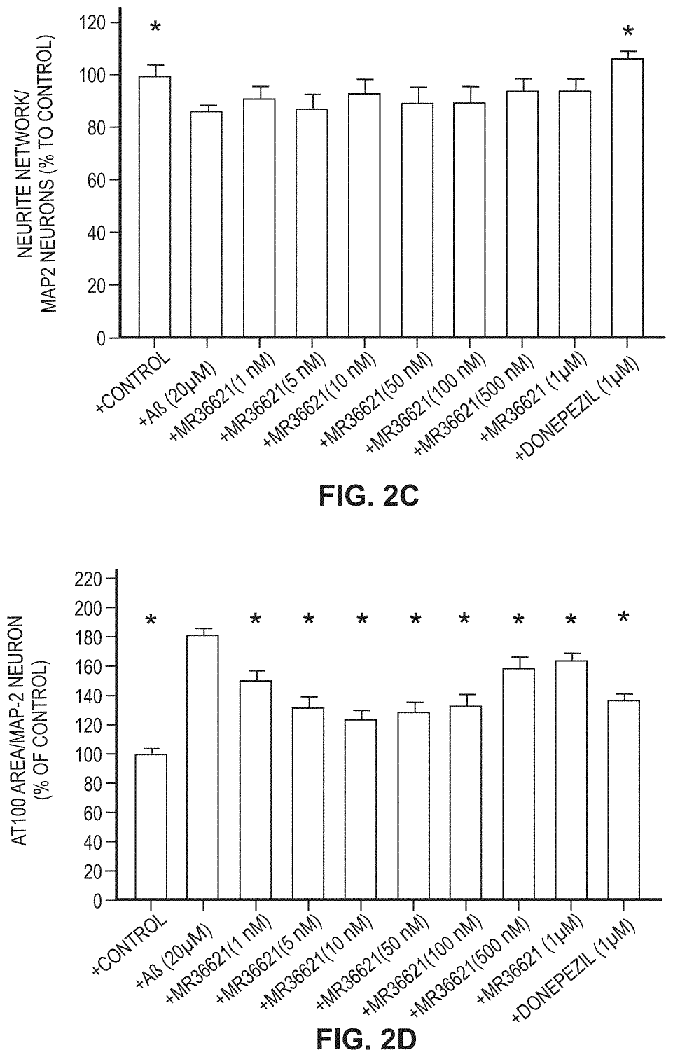

FIGS. 2A-2D are bar graphs that provide the results of the experiments described above. In each of the bar graphs the “*” denotes p<0.05 vs. Aβ; One-way ANOVA (analysis of variance) followed by Fisher's LSD (least significant digit) Test.

Neuronal survival. An important loss of MAP-2 neurons was observed after the application of the Aβ peptide (FIG. 2A). Donepezil, used here as a positive control, was able to significantly protect neurons from death. Low doses of bambuterol (1 nM to 100 nM) exerted significant neuroprotective effects to a similar or higher extent to donepezil. The maximal effect was observed at a dose of 10 nM. The highest doses (500 nM and 1 μM) did not show any protective effect, as shown by the bell-shaped curve effect of bambuterol.

Neurite network. Total neurite network was severely shrunk upon Aβ 1-42 injury (FIG. 2B). Donepezil significantly preserved almost the whole neurite network. All investigated doses of bambuterol exerted protective effects on the neurite network. Of note, the protective effect on the neurite followed a bell-shape curve, as observed on FIG. 2A. The maximal effect was observed at a dose of 10 nM.

The neurite network is proportional to the number of neurons in the culture. The total neurite network was normalized by the number of neurons, in order to determine the average length of neurite per neurons (FIG. 2C). This adjustment showed that neurite length per neuron was longer in presence of donepezil. Bambuterol did not change this parameter.

Tauphosphorylation. An hyperphosphorylation of Tau (on AT100) was observed under Aβ 1-42 application (FIG. 2D). Donepezil was able to significantly mitigate this hyperphosphorylation. Similarly, bambuterol (all investigated doses) significantly reduced the hyperphosphorylation of Tau. Again, this effect followed a bell-shape curve, with a maximal effect at a dose of 10 nM.

Effects of Bambuterol on Synapses and Neurite Network of MAP-2 Hippocampal Neurons and on the Hyperphosphorylation of Tau.

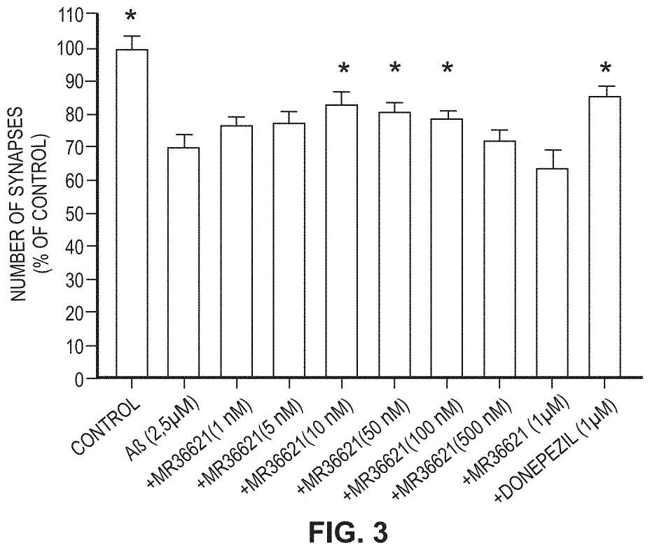

To assess the formation of synapses, the distribution of PSD-95, a post-synaptic marker, and synaptophysin, a pre-synaptic marker, was studied. FIG. 3 shows a structure positive for both PSD-95 and synaptophysin staining was considered as a synapse. The stress resulting from the application of Aβ 1-42 caused a reduction in the number of synapses (as previously shown by Callizot et al., 2013). Donepezil mitigated the loss of synapses. Similarly, bambuterol (from 10 nM to 100 nM) was able to preserve the number of synapses.

The examples show that bambuterol had beneficial effects on the neuronal survival and neurite network of hippocampal neurons injured with Aβ 1-42 peptide, an in vitro model of Alzheimer disease. Moreover, bambuterol was able to reduce the hyperphosphorylation of Tau on the site AT100 and was able to significantly preserve synapses from the Aβ 1-42 injury. The effects of bambuterol (at a dose of 10 nM) were comparable, and even greater, to those of Donepezil.

1-21. (canceled)

22. A method of preventing, managing, or treating Alzheimer's disease comprising: administering a formulation comprising a therapeutically effective amount of bambuterol or a pharmaceutically acceptable salt thereof.

23. The method of claim 22, wherein the bambuterol or the pharmaceutically acceptable salt thereof is encapsulated in liposomes or mixed with phospholipids or micelles.

24. The method of claim 23, wherein the bambuterol or the pharmaceutically acceptable salt thereof is encapsulated in the liposomes to form an encapsulated therapeutic delivery composition.

25. The method of claim 24, wherein the encapsulated therapeutic delivery composition is integrated in a gel.

26. The method of claim 25, wherein the gel is a thermoresponsive gel.

27. The method of claim 26, wherein the thermoresponsive gel is mucoadhesive.

28. The method of claim 22, wherein the bambuterol or the pharmaceutically acceptable salt thereof is present in an amount of between 1 mg and 50 mg.

29. The method of claim 25, wherein said gel is administered to a subject daily, weekly, biweekly or monthly.

30. The method of claim 22, wherein the bambuterol or the pharmaceutically acceptable salt thereof is not encapsulated and is located in a gel.

31. The method of claim 30, wherein the gel is thermoresponsive.

32. The method of claim 31, wherein the thermoresponsive gel is mucoadhesive.

33. The method of claim 30, wherein the bambuterol or the pharmaceutically acceptable salt thereof is present in an amount of between 1 mg and 50 mg.

34. The method of claim 33, wherein said gel is administered to a subject daily, weekly, biweekly or monthly.

35. The method of claim 22 further comprising reducing, hyperphosphorylation of Tau.

36. The method of claim 22 further comprising increasing neuronal survival.

37. The method of any of claim 22 further comprising increasing protection of a neurite network.

38. The method claim 22, wherein said administering is orally.

39.-53. (canceled)

54. The method according to claim 22, wherein the said administering is intranasally.

55. The method according to claim 22, wherein said administering is intravenously.

56. The method of claim 23, wherein the bambuterol or the pharmaceutically acceptable salt thereof is mixed with phospholipids or micelles.
